(19) United States
(12) Patent Application Publication
(10) Pub. No.: US 2022/0346427 A1
(43) Pub. Date: Nov. 3, 2022

(54) SUGAR-RESTRICTED, HIGH-FAT DIET FOR IMPROVING CHRONIC KIDNEY DISEASE

(71) Applicant: OSAKA UNIVERSITY, Osaka (JP)

(72) Inventors: Keisuke HAGIHARA, Osaka (JP); Katsufumi KAJIMOTO, Osaka (JP)

(73) Assignee: OSAKA UNIVERSITY, Osaka (JP)

(21) Appl. No.: 17/763,366

(22) PCT Filed: Sep. 25, 2020
(86) PCT No.: PCT/JP2020/036162

§ 371 Date: Mar. 24, 2022

(30) Foreign Application Priority Data
Sep. 27, 2019 (JP) 2019-178003

(51) Int. Cl. A23L 33/12; A23L 33/17; A23L 33/00

(52) U.S. Cl. CPC A23L 33/12; A23L 33/17; A23L 33/30; A23V 2002/00

(57) ABSTRACT

The present invention provides a sugar-restricted, high-fat diet for improving chronic kidney disease. In the sugar-restricted, high-fat diet, daily fat intake may be set at 120 g or more based on a real body weight of 50 kg or set at an amount corresponding to 70% or more of the total daily energy intake. The sugar-restricted, high-fat diet can increase glomerular filtration rate and reduce blood creatinine level in chronic kidney disease patients.

### TECHNICAL FIELD

The present invention relates to a sugar-restricted, high-fat diet for improving chronic kidney disease.

### BACKGROUND ART

A “ketogenic diet” is one of the well-known dietary regimens. The “ketogenic diet” is a sugar-restricted, high-fat diet characterized in that 60 to 90% of energy intake is from fat. The “ketogenic diet” is therefore used for the treatment of patients who require a sugar-restricted diet, for example, children with epilepsy (e.g., Patent Literature 1). Recently, a dietary regimen based on the “ketogenic diet” has been proposed as a potential treatment for cancer patients (e.g., Patent Literature 2). There are some case reports showing a dramatic clinical response to a ketogenic diet regimen.

Chronic kidney disease (CKD) is defined as a condition in which some kind of kidney damage persists for 3 months or more, and CKD includes all kidney diseases that have a chronic course. There are various causes of CKD. In particular, lifestyle-related diseases (diabetes, hypertension, etc.) and chronic nephritis are the most common causes, and CKD is closely related to metabolic syndrome as well. In Japan, an estimated 13.3 million people (1 out of 8 adults aged 20 and over) suffer from CKD, which is considered to be a new national disease. CKD can progress to renal failure, and when renal failure has occurred, dialysis is required to replace the lost renal function. The number of dialysis patients paying high medical expenses is increasing in Japan with the aging of the population and the increase in lifestyle-related diseases. However, there is still no effective treatment for CKD, and currently, conservative treatment such as blood pressure control, blood glucose control, dietary protein restriction, and dietary salt reduction must be resorted to. There is no single treatment for CKD that can provide active nephroprotection.

### CITATION LIST

### Patent Literature

Patent Literature 1: Japanese Patent No. 5937771
Patent Literature 2: WO 2017/038101

### SUMMARY OF INVENTION

### Technical Problem

An object of the present invention is to find a new target disease for which a ketogenic diet (sugar-restricted, high-fat diet) regimen is effective.

### Solution to Problem

The present invention includes the following to achieve the above-mentioned object.

[1] A sugar-restricted, high-fat diet for improving chronic kidney disease.

[2] The sugar-restricted, high-fat diet according to the above [1], wherein the improving of chronic kidney disease is associated with an increased glomerular filtration rate in a chronic kidney disease patient.

[3] The sugar-restricted, high-fat diet according to the above [1] or [2], wherein the improving of chronic kidney disease is associated with a reduced blood creatinine level in a chronic kidney disease patient.

[4] The sugar-restricted, high-fat diet according to any one of the above [1] to [3], wherein daily fat intake in the diet is set at 120 g or more based on a real body weight of 50 kg or set at an amount corresponding to 70% or more of a total daily energy intake.

[5] The sugar-restricted, high-fat diet according to any one of the above [1] to [4], wherein the fat is a fat containing a medium-chain fatty acid oil.

[6] The sugar-restricted, high-fat diet according to the above [5], wherein the percentage of the medium-chain fatty acid oil in the fat is 30% by mass or more.

[7] The sugar-restricted, high-fat diet according to any one of the above [1] to [6], wherein daily sugar intake in the diet is set at 30 g or less based on a real body weight of 50 kg.

[8] The sugar-restricted, high-fat diet according to any one of the above [1] to [7], wherein the sugar is a sugar containing lactose.

[9] The sugar-restricted, high-fat diet according to any one of the above [1] to [8], wherein the diet comprises 5 to 40% by mass protein.

[10] The sugar-restricted, high-fat diet according to any one of the above [1] to [9], wherein daily calorie intake in the diet is 1000 kcal or more based on a real body weight of 50 kg.

[11] The sugar-restricted, high-fat diet according to any one of the above [1] to [10], wherein the diet comprises 25 to 40% by mass long-chain fatty acid oil, 30 to 50% by mass medium-chain fatty acid oil, 15% by mass or less sugar, and 10 to 30% by mass protein.

### Advantageous Effects of Invention

The present invention provides a sugar-restricted, high-fat diet for improving chronic kidney disease. The sugar-restricted, high-fat diet (ketogenic diet) can increase glomerular filtration rate and reduce blood creatinine level in chronic kidney disease patients.

### BRIEF DESCRIPTION OF DRAWINGS

FIG. 1 shows a change in blood acetoacetic acid levels in 37 analyzed patients who participated in the clinical study of a ketogenic diet regimen for cancer treatment.

FIG. 2 shows a change in blood β-hydroxybutyric acid levels in 37 analyzed patients who participated in the clinical study of a ketogenic diet regimen for cancer treatment.

FIG. 3 shows changes in renal function test values in 37 analyzed patients who participated in the clinical study of a ketogenic diet regimen for cancer treatment. FIG. 3A is a graph of BUN, FIG. 3B is a graph of creatinine, FIG. 3C is a graph of uric acid, and FIG. 3D is a graph of eGFR.

FIG. 4 shows a change in blood acetoacetic acid level in a patient who had a significant improvement in renal function in response to a ketogenic diet regimen for cancer treatment.

FIG. 5 shows a change in blood β-hydroxybutyric acid level in a patient who had a significant improvement in renal function in response to a ketogenic diet regimen for cancer treatment.

FIG. 6 shows a change in blood creatinine level in a patient who had a significant improvement in renal function in response to a ketogenic diet regimen for cancer treatment.

FIG. 7 shows a change in eGFR in a patient who had a significant improvement in renal function in response to a ketogenic diet regimen for cancer treatment.

### DESCRIPTION OF EMBODIMENTS

The present invention provides a sugar-restricted, high-fat diet for improving chronic kidney disease. The “sugar-restricted, high-fat diet” means a diet that restricts sugar intake and allows more fat intake as compared to a normal diet. The “sugar-restricted, high-fat diet” is also known as a “ketogenic diet”. As used herein, the “sugar” refers to a type of carbohydrate that is not a dietary fiber. As used herein, the “carbohydrate” refers to an organic compound composed of a monosaccharide as a structural unit. The “carbohydrate” used in the context of the sugar-restricted, high-fat diet refers to a type of carbohydrate other than a dietary fiber, i.e., a “sugar”, and the terms “carbohydrate” and “sugar” are used interchangeably.

As used herein, the “high-fat diet” is characterized in that daily fat intake in the diet is set at an amount corresponding to about 30% or more of the total daily energy intake. The lower limit of the daily fat intake in the “high-fat diet” may be about 40%, about 45%, about 50%, about 55%, about 60%, about 65%, about 70%, about 75%, about 80%, about 85%, or about 90% of the total daily energy intake. The upper limit may be about 95%, about 90%, about 85%, about 80%, about 75%, about 70%, about 65%, about 60%, about 55%, or about 50% of the total daily energy intake. Any combination of these lower and upper limit values can be used. As used herein, the percent energy from fat is calculated based on 9 kcal/g for fat.

Alternatively, the “high-fat diet” as used herein is characterized in that daily fat intake in the diet is set at about 80 g or more based on a real body weight of 50 kg. The lower limit of the daily fat intake in the “high-fat diet” may be about 90 g, about 100 g, about 110 g, about 115 g, about 120 g, about 125 g, about 130 g, about 135 g, about 140 g, about 145 g, or about 150 g. The upper limit may be about 180 g, about 170 g, about 160 g, about 150 g, or about 140 g. Any combination of these lower and upper limit values can be used.

The fat in the high-fat diet may be a short-chain fatty acid oil, a medium-chain fatty acid oil, a long-chain fatty acid oil, or any combination thereof. The fat in the high-fat diet preferably comprises a high percentage of medium-chain fatty acid oil. The lower limit of the percentage of the medium-chain fatty acid oil in the fat may be about 10%, about 20%, about 30%, about 40%, about 50%, about 60%, about 70%, or about 80%. The upper limit may be about 90%, about 80%, about 70%, or about 60%. Any combination of these lower and upper limit values can be used.

The medium-chain fatty acid oil is an oil composed of fatty acids of medium length and is also referred to as MCT (medium chain triglyceride). Typically, the medium-chain fatty acid oil is composed of fatty acids each having 6 to 12 carbon atoms, preferably 8 to 12 carbon atoms, 8 to 11 carbon atoms, or 8 to 10 carbon atoms. The medium-chain fatty acid oil is more easily digestible and absorbable than commonly used oils and is more readily convertible to energy. Examples of the medium-chain fatty acid include hexanoic acid (caproic acid; C6), octanoic acid (caprylic acid; C8), nonanoic acid (pelargonic acid; C9), decanoic acid (capric acid; C10), and dodecanoic acid (lauric acid; C12).

The medium-chain fatty acid oil is present in fats and oils contained in plants such as coconut, palm fruit, and other palm plants and in milk and other dairy products. The medium-chain fatty acid oil can be extracted (or crudely extracted) or purified (or crudely purified) from these fats and oils (preferably vegetable fats and oils such as palm kernel oil) and used as it is or as a raw material. Alternatively, the medium-chain fatty acid oil may be a chemical synthetic product or a commercial product. For example, Nisshin MCT Oil & Powder (manufactured by Nisshin Oillio) and Extra Virgin Coconut Oil (manufactured by Nisshin Oillio) can be used as the medium-chain fatty acid oil.

As used herein, the “sugar-restricted” means that daily sugar intake is restricted to about 100 g or less based on a real body weight of 50 kg. This value is calculated based on the description in the “Dietary Reference Intakes for Japanese” (2015 edition) published by the Ministry of Health, Labor and Welfare, which states, “If the basal metabolic rate is 1,500 kcal/day, the energy consumption of the brain would be 300 kcal/day, which is equal to 75 g/day of glucose. Since tissues other than the brain also use glucose as an energy source as explained above, the glucose requirement is estimated to be at least 100 g/day. In other words, the minimum requirement for digestible carbohydrates is estimated to be about 100 g/day.”; and therefore, it will be understood that the value of the daily sugar intake specified above can vary. The upper limit of the daily sugar intake in the “sugar-restricted diet” may be about 90 g, about 80 g, about 70 g, about 60 g, about 50 g, about 40 g, about 35 g, about 30 g, about 25 g, about 20 g, about 15 g, or about 10 g. The lower limit may be about 5 g, about 10 g, about 15 g, about 20 g, about 25 g, about 30 g, or about 60 g. Any combination of these lower and upper limit values can be used.

In the sugar-restricted, high-fat diet of the present invention, sugar intake may be set lower during the introduction phase, and for example, may be restricted to about 20 g/day or less or about 10 g/day or less. A lower sugar intake during the introduction phase can induce a rapid increase in blood ketone bodies (acetoacetic acid and β-hydroxybutyric acid). However, such a diet during the introduction phase differs from original dietary habits. In order to continue the sugar-restricted, high-fat diet and thus achieve therapeutic benefits therefrom, gradually relaxing the restriction of sugar intake is helpful. For example, sugar intake may be set at about 5 to 15 g/day or thereabouts (±about 5 g/day) in the introduction phase, maintained at about 15 to 25 g/day or thereabouts (±about 5 g/day) in the second phase, and then maintained at about 25 to 35 g/day or thereabouts (±about 10 g/day) in the subsequent maintenance phase.

The sugar-restricted, high-fat diet may comprise a monosaccharide such as glucose, fructose, or galactose, a disaccharide such as maltose, sucrose, or lactose, or a polysaccharide such as starch (amylose, amylopectin), glycogen, or dextrin, or any combination thereof within the scope of sugar restriction (e.g., within the range of the daily sugar intake described above). The sugar-restricted, high-fat diet of the present invention may optionally be free of glucose or polysaccharides composed of glucose as a basal structural unit. The sugar-restricted, high-fat diet of the present invention preferably comprises lactose (milk sugar), more preferably comprises lactose (milk sugar) without glucose, and particularly preferably comprises substantially only lactose (milk sugar) as a sugar within the scope of sugar restriction (e.g., within the range of the daily sugar intake described above).

The sugar-restricted, high-fat diet of the present invention may comprise a protein. The lower limit of the percentage of the protein in the sugar-restricted, high-fat diet of the present invention may be about 5% by mass or about 10% by mass. The upper limit may be about 40% by mass, about 30% by mass, or about 20% by mass. Any combination of these lower and upper limit values can be used.

Daily calorie intake in the sugar-restricted, high-fat diet of the present invention is preferably, but not limited to, about 20 kcal/kg or more (about 1000 kcal or more for a standard body weight of 50 kg). For example, it may be about 14 kcal/kg/day or more, about 16 kcal/kg/day or more, or about 18 kcal/kg/day or more. Preferably, it is about 22 kcal/kg/day or more, about 24 kcal/kg/day or more, about 26 kcal/kg/day or more, about 28 kcal/kg/day or more, or about 30 kcal/kg/day or more.

The ketone ratio (lipid/(protein+sugar)) (mass ratio) in the sugar-restricted, high-fat diet of the present invention is preferably about 1 or more (rich in lipid). For example, the ketone ratio may be about 2 or more or about 2.5 or more. The upper limit of the ketone ratio can be, for example, about 4 or about 3.5. The ketone ratio during the introduction phase is preferably about 2. The combined intakes of protein and sugar in the sugar-restricted, high-fat diet may be any amount as long as the ketone ratio meets the above-described level. The combined intakes of protein and sugar in the sugar-restricted, high-fat diet may be about 30 g/day or less, about 20 g/day or less, or about 10 g/day or less. The combined intakes of protein and sugar can be varied according to the time of year. The combined intakes of protein and sugar per meal may be any amount as long as they are within the combined intakes of protein and sugar per day, but are preferably about 10 g or less per meal.

A preferable example of the sugar-restricted, high-fat diet of the present invention is a sugar-restricted, high-fat diet comprising about 25 to 40% by mass long-chain fatty acid oil, about 30 to 50% by mass medium-chain fatty acid oil, about 0 to 15% by mass sugar, and about 10 to 30% by mass protein.

The sugar-restricted, high-fat diet may be the Atkins diet when used for children, or the modified Atkins diet when used for adults. The modified Atkins diet is as follows.

(1) During the first week, calorie intake is set at about 30 kcal/kg body weight on a real body weight basis, lipid and protein intakes are unrestricted, and the target for sugar (a type of carbohydrate other than a dietary fiber) intake is set at about 10 g or less. Specifically, when the real body weight is 50 kg, calorie intake is set at about 1500 kcal/day, and the lipid:protein:sugar intake ratio is about 140 g:about 60 g:about 10 g per day in the introduction phase. The target for the ketone ratio (lipid/(protein+sugar)) is set at 2. Other nutrients may be consumed without restriction. Essential trace elements and vitamins are taken in supplements or other forms as needed. The period can be extended or shortened as needed and may range from several days to several weeks.

(2) From the second week to the third month, sugar intake and medium-chain fatty acid intake via a ketogenic formula and MCT oil are adjusted with reference to blood ketone body levels. For example, the targets for acetoacetic acid and β-hydroxybutyric acid levels are set at 500 μmol/L or more and 1000 μmol/L or more, respectively, and if possible, to 1000 μmol/L or more and 2000 μmol/L or more, respectively. Sugar intake is set at about 20 g/day or less, and calorie intake is set at about 1400 to 1600 kcal/day. The lipid:protein:sugar intake ratio is set at about 120 to 140 g:about 70 g:about 20 g per day, and the target for the ketone ratio is set at about 1 to 2. For caloric supplementation, MCT oil and a ketogenic formula can preferably be used. The period can be extended or shortened as needed. The starting point may be shortly before or after the start of the second week, and the end point may be shortly before or after the end of the third month (the schedule may be shifted by one, two, or several weeks).

(3) After the third month, sugar intake is set at 10 g per meal and to about 30 g or less per day, and the others are set basically in the same manner as in the above (2).

The sugar-restricted, high-fat diet can be provided in the form of an appropriate combination of a main dish, a side dish, a soup, etc. This means that the sugar-restricted, high-fat diet of the present invention can be provided in the form of a home delivery meal, a home delivery meal box, a frozen meal box, etc. In addition, the sugar-restricted, high-fat diet can be provided in the form of a sugar-restricted, high-fat meal kit that includes a set of ingredients for a main dish, a side dish, a soup, etc., and a cooking recipe. Furthermore, the sugar-restricted, high-fat diet can be provided in the form of a frozen food, a dairy product, a chilled food, a nutritional food, a liquid food, a nursing food, a beverage, etc.

The present invention provides a sugar-restricted, high-fat composition for improving chronic kidney disease. The sugar-restricted, high-fat composition of the present invention can be used in such a manner as to meet the intake (fat intake, sugar intake, protein intake, calorie intake, etc.) levels required by the sugar-restricted, high-fat diet of the present invention described above.

Preferable examples of the sugar-restricted, high-fat composition of the present invention include a ketogenic formula (817-B; Meiji Co., Ltd.), compositions equivalent in ingredient composition to this formula, and modified compositions of the formula. The modified compositions of the ketogenic formula include, for example, compositions that are the same as the ketogenic formula (817-B) except for reduced amounts of sugar and/or protein, and compositions that are the same as the ketogenic formula (817-B) except that the amounts of the ingredients are independently varied by ±about 5%, ±about 10%, ±about 15%, ± about 20%, or ±about 25%. The ingredient composition of the ketogenic formula is shown in

Tables 1 and 2.

TABLE 1
Ingredient composition of ketogenic formula (817-B) (1)

| | Amount per 100 g of | |
|---|---|---|
| Ingredients | ketogenic formula (% E) | |
| Protein | 15.0 | g (8.1) |
| Lipid | 71.8 | g (87.2) |
| Carbohydrate | 8.8 | g (4.7) |
| Ash | 2.4 | g (0) |
| Water | 2.0 | g (0) |
| Energy | 741 | kcal |

Notes:
Protein: Lactoprotein
Lipid: Long-chain fatty acid fat and oil (essential fatty acid-modified fat) 32.1 g (39.0% E)
Medium-chain fatty acid fat and oil 39.7 g (48.2% E)
Carbohydrate: Lactose
% E: % Energy

TABLE 2
Ingredient composition of ketogenic formula (817-B) (2)

| | Amount per 100 g of | |
|---|---|---|
| Ingredients | ketogenic formula | |
| Vitamin A | 600 | μg RE |
| Vitamin B1 | 0.6 | mg |
| Vitamin B2 | 0.9 | mg |
| Vitamin B6 | 0.3 | mg |
| Vitamin B12 | 4 | μg |
| Vitamin C | 50 | mg |
| Vitamin D | 12.5 | μg |
| Vitamin E | 6 | mg α-TE |
| Vitamin K | 30 | μg |
| Pantothenic acid | 2 | mg |
| Niacin | 6 | mg |
| Folic acid | 0.2 | mg |
| Calcium | 350 | mg |
| Magnesium | 36 | mg |
| Sodium | 165 | mg |
| Potassium | 470 | mg |
| Phosphorus | 240 | mg |
| Chlorine | 320 | mg |
| Iron | 6 | mg |
| Copper | 350 | μg |
| Zinc | 2.6 | mg |

The sugar-restricted, high-fat composition of the present invention can be used as the sugar-restricted, high-fat diet of the present invention (preferably the modified Atkins diet).

The sugar-restricted, high-fat diet and sugar-restricted, high-fat composition of the present invention can be used for improving chronic kidney disease (CKD). Chronic kidney disease is a condition in which some kind of kidney damage persists for 3 months or more. The presence of kidney damage is determined based on pathological diagnosis, diagnostic imaging, urinalysis (e.g., proteinuria), blood tests (e.g., creatinine, BUN), estimated glomerular filtration rate (eGFR), etc. Chronic kidney disease is caused by a variety of factors, including aging; lifestyle-related diseases such as diabetes, hypertension, dyslipidemia, and hyperuricemia; autoimmune diseases such as chronic nephritic syndrome and collagen disease; urological diseases such as urolithiasis; adverse reactions to drugs (e.g., non-steroidal anti-inflammatory analgesics etc.); and heredity (e.g., resulting in polycystic kidney disease etc.).

As shown in the Examples below, according to the present inventors' study, a kidney cancer patient who had undergone total left and partial right nephrectomy was placed on the sugar-restricted, high-fat diet of the present invention, and after three months from the start of the diet, the creatinine level was significantly reduced from the level before the start of the diet, and eGFR was significantly increased from the level before the start of the diet. At the time of filing this application, there are still no reports of medicines that can increase eGFR. For example, an article by Perkovic, J., et al. (N Engl J Med 2019; 380:2295-2306) reports a double-blind randomized trial of canagliflozin (an oral SGLT2 inhibitor) in patients with type 2 diabetes and kidney disease, in which trial the risk of renal failure and cardiovascular events was significantly reduced in the patients treated with canagliflozin. However, as shown in FIG. 3B of this article, although eGFR reduction was suppressed inpatients treated with canagliflozin as compared to that in patients treated with placebo, eGFR was not increased. Therefore, the effect of the present invention, i.e., a significant increase in eGFR demonstrated by the present inventors, would have been completely unpredictable to those skilled in the art.

The present invention further includes the following.

A method for improving chronic kidney disease, comprising placing a chronic kidney disease patient on a sugar-restricted, high-fat diet.

A sugar-restricted, high-fat diet for use in improving chronic kidney disease.

Use of a sugar-restricted, high-fat diet for improving chronic kidney disease.

A sugar-restricted, high-fat diet for increasing glomerular filtration rate in a chronic kidney disease patient.

A method for increasing glomerular filtration rate in a chronic kidney disease patient, comprising placing the patient on a sugar-restricted, high-fat diet.

A sugar-restricted, high-fat diet for use in increasing glomerular filtration rate in a chronic kidney disease patient.

Use of a sugar-restricted, high-fat diet for increasing glomerular filtration rate in a chronic kidney disease patient.

### EXAMPLES

Hereinafter, the present invention will be described in detail by examples, but the present invention is not limited thereto.

### Example 1: Clinical Study of Ketogenic Diet Regimen for Cancer Treatment

1-1 Test method

(1) Participants

Patients who had stage IV cancer, a performance status (PS) of 2 or lower, and were capable of oral ingestion were included in the study. Fifty-five cancer patients (24 men and 31 women) participated in the study. The average age of the participants was 55.8±12.1 years. Forty-two of the participants had received chemotherapy, 32 had received surgery, and 17 had received radiation therapy.

(2) Ketogenic Diet

From the initial time until one week later, calorie intake was set at 30 kcal/kg/day on a real body weight basis. Lipid and protein intakes were unrestricted, and the target for carbohydrate (a type of carbohydrate other than a dietary fiber, which is equivalent to sugar, and the same applies hereinafter) intake was set at 10 g/day or less. For example, for a real body weight of 50 kg, calorie intake was set at 1500 kcal/day, and the lipid:protein:sugar intake ratio was set at 140 g:60 g:10 g per day. The target for the ketone ratio [lipid (g): (protein (g)+carbohydrate (g))] was set at 2:1. Other nutrients were allowed to be consumed without restriction. Essential trace elements and vitamins were taken in supplements or other forms as needed. In the introduction phase, the participants took meals prepared according to the ketogenic diet menu provided by dietitians.

From one week to three months after the start of the ketogenic diet, the contents of the meals were determined with reference to blood ketone body measurements. Regarding blood ketone body levels, the targets for acetoacetic acid and β-hydroxybutyric acid levels were set at 500 μmol/L or more and 1000 μmol/L or more, respectively, and if possible, to 1000 μmol/L or more and 2000 μmol/L or more, respectively. Carbohydrate intake was set at 20 g/day or less. For example, for a real body weight of 50 kg, calorie intake was set at 1400 to 1600 kcal/day, and the lipid:protein:sugar intake ratio was set at 120 to 140 g: 70 g:20 g per day. The target for the ketone ratio [lipid (g): (protein (g)+carbohydrate (g))] was set at 2:1 to 1:1. For caloric supplementation, “MCT Oil” (manufactured by Nisshin OilliO) or “Ketonformula” (manufactured by Meiji Co., Ltd.) was used.

(3) Blood Sampling and Biochemical Tests

Blood samples were taken before the start of the ketogenic diet, one week after the start of the ketogenic diet, one month after the start of the ketogenic diet, two months after the start of the ketogenic diet, and three months after the start of the ketogenic diet to measure blood acetoacetic acid, blood β-hydroxybutyric acid, blood urea nitrogen (BUN), creatinine, and uric acid. In addition, an estimated glomerular filtration rate (eGFR) was calculated based on the creatinine level, age, and sex.

Of a total of 55 participants, 5 did not take the ketogenic diet, 11 discontinued the study, and 2 were excluded from the analysis, so that 37 participants (15 males and 22 females) were finally analyzed. The finally analyzed participants had an average age of 54.8±12.6 years, an average body height of 162.5±9.5 cm, an average body weight of 55.5±13.2 kg, and an average BMI of 20.9±3.7 as of the start of the study. Six of the finally analyzed participants had lung cancer, 8 had colorectal cancer, 5 had breast cancer, 1 had ovarian cancer, 1 had bladder cancer, and 16 had other cancers. Thirty-two of the finally analyzed participants had received chemotherapy, 25 had received surgery, and 13 had received radiation therapy.

1-2 Results

(1) Change in Blood Ketone Body Levels

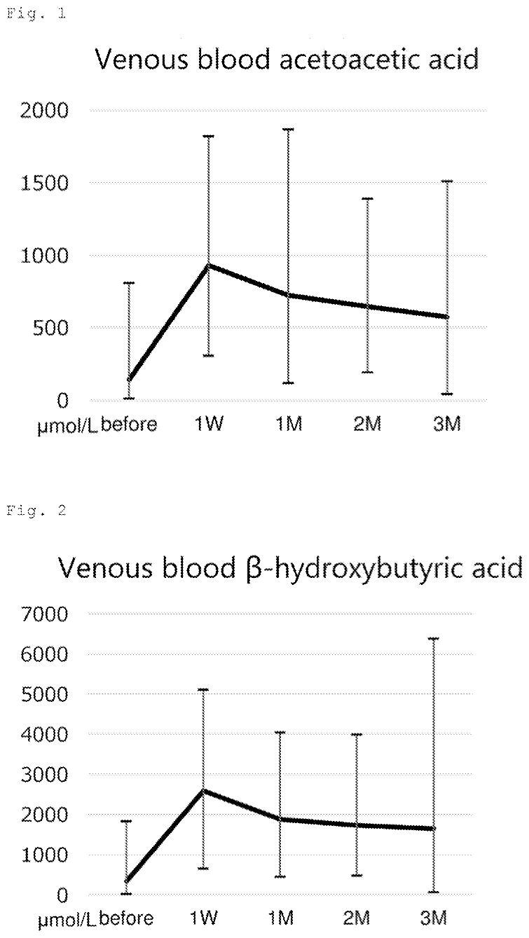

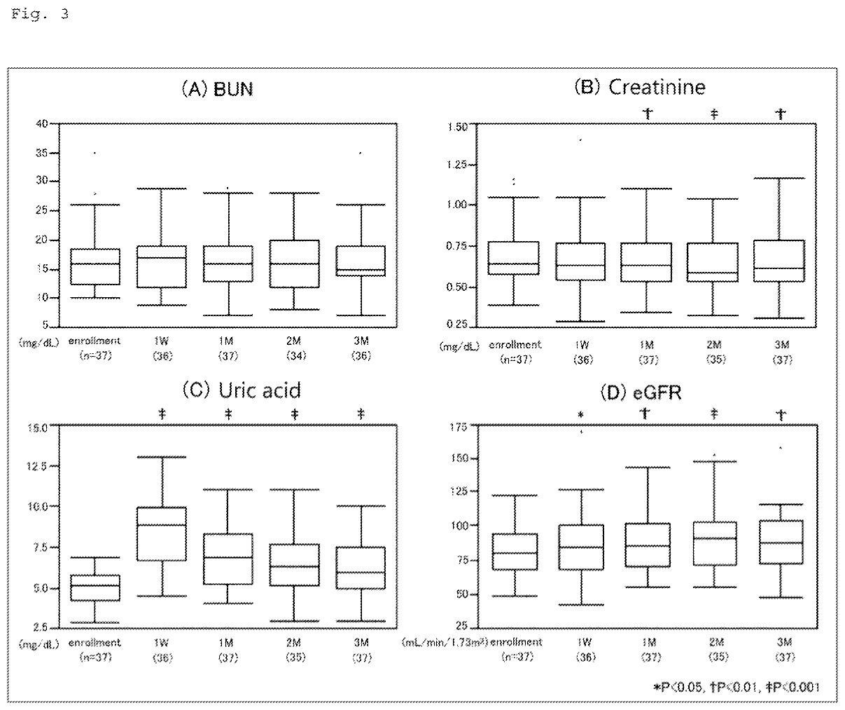

FIG. 1 shows a change in blood acetoacetic acid levels in the analyzed participants from before the start of the ketogenic diet to 3 months after the start of the ketogenic diet. FIG. 2 shows a change in blood β-hydroxybutyric acid levels in the analyzed participants from before the start of the ketogenic diet to 3 months after the start of the ketogenic diet. The average acetoacetic acid level was maintained at 500 μmol/L or more throughout the test period, and the average β-hydroxybutyric acid level was maintained at 1000 μmol/L or more throughout the test period.

(2) Changes in renal function test values FIG. 3 shows changes in renal function test values in the analyzed participants from before the start of the ketogenic diet to 3 months after the start of the ketogenic diet. FIG. 3A shows the results for BUN, FIG. 3B shows the results for creatinine, FIG. 3C shows the results for uric acid, and FIG. 3D shows the results for eGFR. The creatinine level after 3 months was significantly lower than that at the start of the study, and the eGFR after 3 months was significantly higher than that at the start of the study, demonstrating that the ketogenic diet is effective in improving renal function. The significantly higher uric acid levels after the start of the ketogenic diet are due to the diet and are not related to renal function.

### Example 2: Case report of significant response

Data from a patient who had a significant improvement in renal function in response to a ketogenic diet regimen for cancer treatment are shown below.

2-1 Medical History

The patient was a 58-year-old man with kidney cancer (body weight: 64.6 kg, body fat percentage: 23.9%). He was diagnosed with kidney cancer in May 2011 and underwent total left and partial right nephrectomy on May 12, 2011. In 2013, right lung metastasis (S10) was detected, and thoracoscopic surgery was performed in November. In 2016, metastases to the 1 lth thoracic vertebra were detected, and laminectomy was performed. Oral treatment with Votrient was started in 2017. Nivolumab infusion was performed in 2018. In the same year, metastases to the 8th thoracic vertebra to the left 7th rib were detected, and cryotherapy was started. In 2019, metastases to the left 5th rib were detected, and stereotactic radiotherapy was performed. In the same year, metastases to the bilateral hilar lymph nodes, mediastinal lymph node, pancreas, and subcutis were detected. Subsequently, a ketogenic diet regimen was started.

2-2 Results

(1) Change in Blood Ketone Body Level

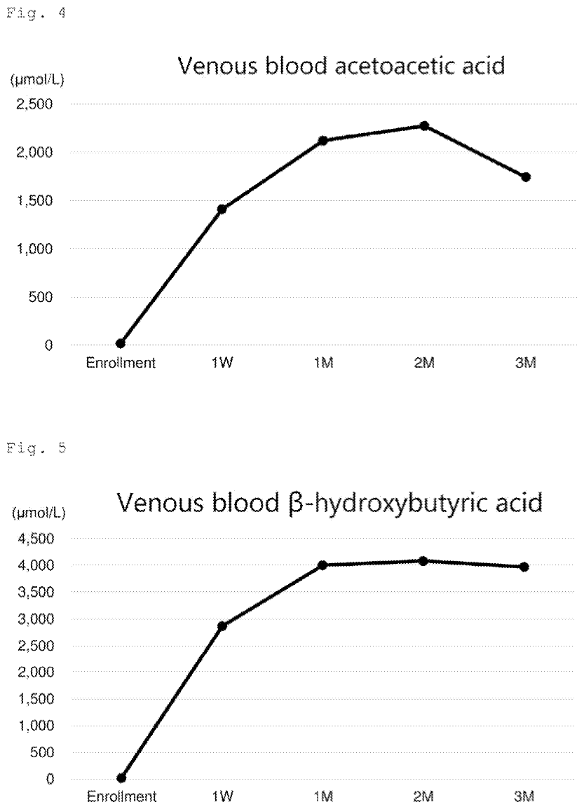

FIG. 4 shows a change in blood acetoacetic acid level in the patient from before the start of the ketogenic diet to 3 months after the start of the ketogenic diet. FIG. 5 shows a change in blood β-hydroxybutyric acid level in the patient from before the start of the ketogenic diet to 3 months after the start of the ketogenic diet. The acetoacetic acid level was 2000 μmol/L or more one and two months later, and about 1700 μmol/L three months later. The β-hydroxybutyric acid level was maintained at about 4000 μmol/L from 1 month to 3 months after the start of the ketogenic diet.

(2) Changes in Renal Function Test Values

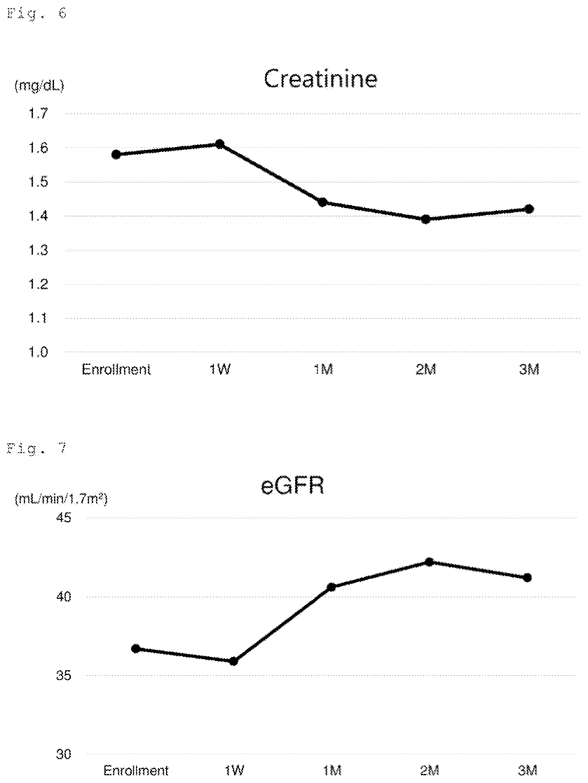

FIG. 6 shows a change in creatinine level in the patient from before the start of the ketogenic diet to 3 months after the start of the ketogenic diet. FIG. 7 shows a change in eGFR in the patient from before the start of the ketogenic diet to 3 months after the start of the ketogenic diet. The creatinine level decreased and eGFR increased after the start of the ketogenic diet, demonstrating that the ketogenic diet regimen provided a significant improvement in renal function in the patient who had undergone total left and partial right nephrectomy.

The present invention is not limited to the particular embodiments and examples described above, and various modifications can be made within the scope of the appended claims. Other embodiments provided by suitably combining technical means disclosed in separate embodiments of the present invention are also within the technical scope of the present invention.

1-11. (canceled)

12. A method for improving chronic kidney disease, comprising placing a chronic kidney disease patient on a sugar-restricted, high-fat diet.

13. The method according to claim 12, wherein the improving of chronic kidney disease is associated with an increased glomerular filtration rate in the chronic kidney disease patient.

14. The method according to claim 12, wherein the improving of chronic kidney disease is associated with a reduced blood creatinine level in the chronic kidney disease patient.

15. The method according to claim 12, wherein daily fat intake in the diet is set at 120 g or more based on a real body weight of 50 kg or set at an amount corresponding to 70% or more of a total daily energy intake.

16. The method according to claim 12, wherein the fat is a fat containing a medium-chain fatty acid oil.

17. The method according to claim 16, wherein the percentage of the medium-chain fatty acid oil in the fat is 30% by mass or more.

18. The method according to claim 12, wherein daily sugar intake in the diet is set at 30 g or less based on a real body weight of 50 kg.

19. The method according to claim 12, wherein the sugar is a sugar containing lactose.

20. The method according to claim 12, wherein the diet comprises 5 to 40% by mass protein.

21. The method according to claim 12, wherein daily calorie intake in the diet is 1000 kcal or more based on a real body weight of 50 kg.

22. The method according to claim 12, wherein the diet comprises 25 to 40% by mass long-chain fatty acid oil, 30 to 50% by mass medium-chain fatty acid oil, 15% by mass or less sugar, and 10 to 30% by mass protein.
